(19) United States
(12) Patent Application Publication
(10) Pub. No.: US 2024/0177841 A1
(43) Pub. Date: May 30, 2024

(54) MEDICAL SUPPLY INVENTORY MANAGEMENT SYSTEM

(71) Applicant: Michael Mamaghani, Rancho Santa Fe, CA (US)

(72) Inventor: Michael Mamaghani, Rancho Santa Fe, CA (US)

(21) Appl. No.: 17/646,499

(22) Filed: Dec. 30, 2021

(51) Int. Cl. G16H 40/20; G06Q 10/087; G07C 9/00; G16H 20/10

(52) U.S. Cl. CPC G16H 40/20; G06Q 10/087; G07C 9/00896; G16H 20/10

(57) ABSTRACT

In various aspects, a system includes a container arranged to store inventory items at storage sites within it, each site including a scale to generate a weight for an inventory item. The system can also include a controller coupled to the container and to the scales and configured to determine an identity or weight of any inventory item removed from or placed at the respective storage sites. The system may be configured to enhance security, weight measurement accuracy, and real-time inventory management for the container.

### BACKGROUND

This disclosure relates generally to inventory management systems. More specifically, this disclosure pertains to secure, real-time medical supply inventory management systems enabled with precise detection mechanisms and artificial intelligence.

Systems to manage inventory are generally unsuitable for items such as medical products, e.g., controlled drugs. Most inventory management systems are labor intensive and thus give rise to human errors such as inaccurate reporting of quantities of an item removed and mistaken removal of the wrong item. Such inventory management systems also give rise to delays caused by time to fill orders, inaccurate destinations specified for delivery, and returns of wrongly delivered items. Such delays are unacceptable in the context of controlled drugs.

Removal of the wrong quantity of a drug, or of a different drug from the one that was requested, may also be intentional. For example, the addiction rates of physicians are as high as, or perhaps even higher than, those of the general adult population. Controlled drug reporting is based on an honor system and is prone to abuse. Efforts to apply real-time techniques to inventory management, such as, for example, the system described in U.S. Pat. No. 6,341,271, fail to provide the security needed in a medical products supply environment, as well as failing to recognize and correct human errors.

The above-described background is merely intended to provide a contextual overview of some current issues, and is not intended to be exhaustive.

### BRIEF DESCRIPTION OF THE DRAWINGS

Non-limiting and non-exhaustive embodiments of the subject disclosure are described with reference to the following figures, wherein like reference numerals refer to like parts throughout the various views unless otherwise specified.

FIG. 1 is a block diagram of an inventory management system architecture in accordance with various aspects of the subject disclosure.

FIG. 2 is a flow chart depicting flow and actions in accordance with various aspects of the subject disclosure.

FIG. 3 is a flow chart depicting an anomaly detection process in accordance with various aspects of the subject disclosure.

FIG. 4 is a flow chart depicting a unit count calculation process in accordance with various aspects of the subject disclosure.

FIG. 5 is a flow chart depicting a process for integrating Practice Management Software with scales in accordance with various aspects of the subject disclosure.

FIG. 6 is a flow chart depicting a product setup process in accordance with various aspects of the subject disclosure.

FIG. 7 is a flow chart depicting a system routine self-analysis process in accordance with various aspects of the subject disclosure.

FIG. 8 is a flow chart depicting a scale measurement process in accordance with various aspects of the subject disclosure.

FIG. 9 is a flow chart depicting a scale weight change process in accordance with various aspects of the subject disclosure.

FIG. 10 is a flow chart depicting a smart ordering system process in accordance with various aspects of the subject disclosure.

### DETAILED DESCRIPTION

In the following description, numerous specific details are set forth to provide a thorough understanding of various aspects and arrangements. It will be recognized, however, that the techniques described herein can be practiced without one or more of the specific details, or with other methods, components, materials, etc. In other instances, well known structures, materials, or operations may not be shown or described in detail to avoid obscuring certain aspects.

Reference throughout this specification to “an aspect,” “an arrangement,” “a configuration,” or “an example” indicates that a particular feature, structure, or characteristic is described. Thus, appearances of phrases such as “in one aspect,” or “in one arrangement,” “in a configuration,” “in some examples,” or the like in various places throughout this specification do not necessarily each refer to the same aspect, feature, configuration, example, or arrangement. Furthermore, the particular features, structures, and/or characteristics may be combined in any suitable manner.

To the extent used in the present disclosure and claims, the terms “component,” “system,” “platform,” “layer,” “selector,” “interface,” and the like are intended to refer to a computer-related entity or an entity related to an operational apparatus with one or more specific functionalities, wherein the entity can be either hardware, a combination of hardware and software, software, or software in execution. As an example, a component may be, but is not limited to being, a process running on a processor, a processor, an object, an executable, a thread of execution, a program, and/or a computer. By way of illustration and not limitation, both an application running on a server and the server itself can be a component. One or more components may reside within a process and/or thread of execution and a component may be localized on one computer and/or distributed between two or more computers. In addition, components may execute from various computer-readable media, device-readable storage devices, or machine-readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which may be operated by a software or firmware application executed by a processor, wherein the processor can be internal or external to the apparatus and executes at least a part of the software or firmware application. As yet another example, a component can be an apparatus that provides specific functionality through electronic components without mechanical parts; the electronic components can include a processor therein to execute software or firmware that confers at least in part the functionality of the electronic components.

To the extent used in the subject specification, terms such as “store,” “storage,” “data store,” “data storage,” “database,” and the like refer to memory components, entities embodied in a memory, or components comprising a memory. It will be appreciated that the memory components described herein can be either volatile memory or nonvolatile memory, or can include both volatile and nonvolatile memory.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A, X employs B, or X employs both A and B, then “X employs A or B” is satisfied under any of the foregoing instances. Moreover, articles “a” and “an” as used in the subject disclosure and claims should generally be construed to mean “one or more” unless specified otherwise or clear from context to be directed to a singular form.

The words “exemplary” and/or “demonstrative,” to the extent used herein, mean serving as an example, instance, or illustration. For the avoidance of doubt, the subject matter disclosed herein is not limited by disclosed examples. In addition, any aspect or design described herein as “exemplary” and/or “demonstrative” is not necessarily to be construed as preferred or advantageous over other aspects or designs, nor is it meant to preclude equivalent exemplary structures and techniques. Furthermore, to the extent that the terms “includes,” “has,” “contains,” and other similar words are used in either the detailed description or the claims, such terms are intended to be inclusive, in a manner similar to the term “comprising” as an open transition word, without precluding any additional or other elements.

As used herein, the term “infer” or “inference” refers generally to the process of reasoning about, or inferring states of, the system, environment, user, and/or intent from a set of observations as captured via events and/or data. Captured data and events can include user data, device data, environment data, data from sensors, application data, implicit data, explicit data, etc. Inference can be employed to identify a specific context or action or can generate a probability distribution over states of interest based on a consideration of data and events, for example.

The disclosed subject matter can be implemented as a method, apparatus, or article of manufacture using standard programming and/or engineering techniques to produce software, firmware, hardware, or any combination thereof to control a computer to implement the disclosed subject matter. The term “article of manufacture,” to the extent used herein, is intended to encompass a computer program accessible from any computer-readable device, machine-readable device, computer-readable carrier, computer-readable media, or machine-readable media. For example, computer-readable media can include, but are not limited to, a magnetic storage device, e.g., hard disk; floppy disk; magnetic strip(s); an optical disk (e.g., compact disk (CD), digital video disc (DVD), Blu-ray Disc (BD)); a smart card; a flash memory device (e.g., card, stick, key drive); a virtual device that emulates a storage device; and/or any combination of the above computer-readable media.

Generally, program modules include routines, programs, components, data structures, etc., that perform particular tasks or implement particular abstract data types. The illustrated aspects of the subject disclosure may be practiced in distributed computing environments where certain tasks are performed by remote processing devices that are linked through a communications network. In a distributed computing environment, program modules can be located in both local and remote memory storage devices.

Computing devices can include at least computer-readable storage media, machine-readable storage media, and/or communications media. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data, or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible and/or non-transitory media that can be used to store desired information. In this regard, the terms “tangible” or “non-transitory” herein as applied to storage, memory, or computer-readable media, are to be understood to exclude only propagating transitory signals per se as modifiers, and do not exclude any standard storage, memory, or computer-readable media that are not only propagating transitory signals per se.

Computer-readable storage media can be accessed by one or more local or remote computing devices, e.g., via access requests, queries, or other data retrieval protocols, for a variety of operations with respect to the information stored by the medium.

A system bus, as may be used herein, can be any of several types of bus structure that can further interconnect to a memory bus (with or without a memory controller), a peripheral bus, and a local bus using any of a variety of commercially available bus architectures. A database, as may be used herein, can include basic input/output system (BIOS) that can be stored in a non-volatile memory such as ROM, EPROM, or EEPROM, with BIOS containing the basic routines that help to transfer information between elements within a computer, such as during startup. RAM can also include a high-speed RAM such as static RAM for caching data.

As used herein, a computer can operate in a networked environment using logical connections via wired and/or wireless communications to one or more remote computers. The remote computer(s) can be a workstation, server, router, personal computer, portable computer, microprocessor-based entertainment appliance, peer device or other common network node. Logical connections depicted herein may include wired/wireless connectivity to a local area network (LAN) and/or larger networks, e.g., a wide area network (WAN). Such LAN and WAN networking environments are commonplace in offices and companies, and facilitate enterprise-wide computer networks, such as intranets, any of which can connect to a global communications network, e.g., the Internet.

When used in a LAN networking environment, a computer can be connected to the LAN through a wired and/or wireless communication network interface or adapter. The adapter can facilitate wired or wireless communication to the LAN, which can also include a wireless access point (AP) disposed thereon for communicating with the adapter in a wireless mode.

When used in a WAN networking environment, a computer can include a modem or can be connected to a communications server on the WAN via other means for establishing communications over the WAN, such as by way of the Internet. The modem, which can be internal or external, and a wired or wireless device, can be connected to a system bus via an input device interface. In a networked environment, program modules depicted herein relative to a computer or portions thereof can be stored in a remote memory/storage device.

When used in either a LAN or WAN networking environment, a computer can access cloud storage systems or other network-based storage systems in addition to, or in place of, external storage devices. Generally, a connection between a computer and a cloud storage system can be established over a LAN or a WAN, e.g., via an adapter or a modem, respectively. Upon connecting a computer to an associated cloud storage system, an external storage interface can, with the aid of the adapter and/or modem, manage storage provided by the cloud storage system as it would other types of external storage. For instance, the external storage interface can be configured to provide access to cloud storage sources as if those sources were physically connected to the computer.

As it is employed in the subject specification, the term “processor” can refer to substantially any computing processing unit or device comprising, but not limited to comprising, single-core processors; single-core processors with software multithread execution capability; multi-core processors; multi-core processors with software multithread execution capability; multi-core processors with hardware multithread technology; vector processors; pipeline processors; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a state machine, a discrete gate or transistor logic, discrete hardware components, or any combination thereof designed to perform the functions described herein. Processors can exploit nano-scale architectures such as, but not limited to, molecular and quantum-dot based transistors, switches, and gates, in order to optimize space usage or enhance performance of user equipment. A processor may also be implemented as a combination of computing processing units. For example, a processor may be implemented as one or more processors together, tightly coupled, loosely coupled, or remotely located from each other. Multiple processing chips or multiple devices may share the performance of one or more functions described herein, and similarly, storage may be effected across a plurality of devices. A processor may be implemented to reside in a cloud-based network such as, e.g., the Internet.

As an overview, various arrangements are described herein. For simplicity of explanation, the methods (or algorithms) are depicted and described as a series of steps or actions. It is to be understood and appreciated that the various arrangements are not limited by the actions illustrated and/or by the order of actions. For example, actions can occur in various orders and/or concurrently, and with other actions not presented or described herein. Furthermore, not all illustrated actions may be required to implement the methods. In addition, the methods could alternatively be represented as a series of interrelated states via a state diagram or events. Additionally, the methods described hereafter are capable of being stored on an article of manufacture (e.g., a machine-readable storage medium) to facilitate transporting and transferring such methodologies to computers.

In various non-limiting example aspects, an inventory management system for medical supplies is disclosed that enables real-time inventory management using artificial intelligence and scale measurements. In one example a combination of scale measurements and camera observations can be used. The inventory management system may identify a user who removes an item by any one or more of the following techniques: RFID card reader, facial recognition, keypad usage, wearables (e.g., Apple Watch®), BLUETOOTH®, and/or Near Field Communication (NFC). In an example, the inventory management system may use smart weight scales to determine which item has been removed and the quantity of the item that was removed. If an item is misplaced on an incorrect scale pad after having been removed, the system would detect an error because the weight on the incorrect scale would have changed by an unexpected amount; the system would then alert the user to place the item on the correct scale pad. In an example, one or more cameras can be installed in different locations within an inventory cabinet to detect whether an item is misplaced. The cameras may be used to read product labels and/or scan bar codes for product information. In an example, the inventory management system can eliminate error by comparing a removed item and the quantity of the removed item to prescription information associated with an intended recipient of the item. In an example, the inventory management system may use software to compare reduced weights on each pad or scale in the cabinet to the quantity of the medication prescribed on the prescription and thus learn the weight of each item, pill, or volume (e.g., of vaccine in a vial) over time. By way of non-limiting example, if a scale weight reading is reduced by 2.5 grams and the associated prescription requires 5 pills, the software would determine that the weight of each pill is 0.5 grams. In an example, the inventory management system may use software to determine the amount of an order based on any one or more of past quantity history, seasonal factors, supply shortages, product life, epidemics, and/or price reductions and vendor promotions. The software may then place the order through office management software or directly through a vendor's web portal: In various aspects and examples, an inventory management system may include multiple network-connected weight scales, optionally one or more cameras, a controller to monitor and manage the multiple scales and report to artificial intelligence-based inventory management software, a database to store product counts and information associated with products and recipients, and at least one inventory cabinet. An inventory cabinet may, in some examples, include one or more door locks to maintain inventory cabinet doors in a locked state until a user is identified.

An inventory management system in accordance with various disclosed examples can provide security by identifying a user by any of various techniques such as, by way of non-limiting example, RFID card, facial recognition, keypad (for example, on a tablet such as an iPad® or a tablet running the Android® operating system), wireless proximity detection technologies such as BLUETOOTH® or NFC, longer range wireless technologies such as wireless LAN, e.g., 802.11 standards, and WAN or cellular, including 2G, 3G, 4G, or 5G technologies, and/or a wearable device such as, for example, an Apple Watch® or a watch running the Android® operating system. Such inventory management system may further detect when an item is removed from any pad in a cabinet, down to, for example, an individual pill or a vaccine vial. Such inventory management system may also detect prescription errors by matching removed drugs to prescription data. Such inventory management system may also deploy an artificial intelligence engine or processor, by way of non-limiting example, in software running on a computer, to order new inventory based on, by way of non-limiting example, seasonal factors, past usage or order history, price promotions from different suppliers, supply shortages, extant epidemics, and/or product life or expiration date. Such inventory management system may dramatically improve security (for example, by preventing substance abuse and/or theft by employees) and patient safety (for example, by preventing incorrect medications being given to patients) while reducing capital requirements of inventory (for example, by enabling just-in-time inventory management, eliminating revenue loss due to lack of inventory, and/or enabling lowest price procurement).

In some examples, the utility of pads or scales in an inventory management system may be enhanced. For example, pad or scale sensitivity to detect small changes in weight, such as, by way of non-limiting example, weight changes of 0.1 grams, may be enabled. The change in weight between a first weighing time and a second weighing time may be measured based on a known weight of an item such as a pill in order to account for the scale drifting over time.

In some examples errors by humans, for example employees, may be detected by using an Application Programming Interface (API) from Practice Management Software to define expected behavior and identify when the wrong type of medicine is taken and/or the wrong quantity of pills are removed from a pill bottle, and provide an alarm or an alert in the event.

In various alternative, or additional, aspects, rather than deploying multiple pads or scales, an entire shelf in a cabinet of an inventory management system may be positioned atop one pad or scale, or a combination of the two approaches may be used. One or more cameras may be used in conjunction with the pad or scale supporting the shelf to determine the type of medicine and/or the number of pills removed from a bottle. Additionally or in place of cameras, the system may use weight measurements of multiple scales to triangulate and identify the type of medicine and/or the number of pills removed from a bottle. Additionally or in place of optical cameras, ultrasound cameras, ultrasonic cameras, ultrawideband (UWB), infrared cameras, or other known cameras or sensors may be used to identify the type of medicine and/or the number of pills removed from a bottle. In some examples, additionally or in the alternative to such ultrasound or other such cameras or sensors, pressure sensors or pressure mats may be used, or may be used in combination with electric switches such as miniature snap-action switches, or microswitches. For example, one or multiple pressure sensors may be placed on a shelf, underneath medicine bottles, to detect which medicine bottle was removed from the shelf.

In some aspects a smart ordering system may be deployed to track histories of orders of medicines and/or pricing of various medicine suppliers, etc. to enable a real-time, or just-in-time, ordering system. Trends may be determined and relied upon as appropriate. In a non-limiting example, a previous number of weeks of ordering history, e.g., four or eight weeks, may be used to estimate or predict requirements for the same medicine for a future period of time, e.g., the next two weeks. The efficacy of such a prediction technique may be determined, and the prediction technique approach may be modified or abandoned, by examining how well the prediction technique worked for the medicine for the same time period in a previous year, by way of non-limiting example the immediately previous year.

In some examples a calibration process may be used to calibrate new pads or scales, and/or to verify the accuracy of pads or scales as to error and/or drift over time.

In some examples, which may be particularly useful in the absence of a lockable door on a cabinet in an inventory management system, and which may be used in addition to or as an alternative to a lockable door, humans (e.g., employees) may be identified and/or authenticated by using facial recognition. In the event facial recognition fails or is less accurate due to the employee wearing a face-covering or partial face-covering mask, other techniques such as, e.g., identification or authentication by a QR code on the mask, an RFID tag on the mask, and/or an NFC tag on the mask, may be used in addition to or as alternatives to facial recognition. Accordingly, the need for a door lock mechanism for the cabinet may be reduced such that a door lock mechanism may be an optional feature.

In some examples error correction may be enabled by detecting a measurement or measurements that fall outside a “normal” range, or detecting that a human such as, e.g., an employee who takes a pill is not the appropriate person to have done so. In such events an alarm or alert may be generated. The need for a door lock mechanism for a cabinet in an inventory management system may be reduced such that a door lock mechanism may be an optional feature.

In various non-limiting aspects, a smart ordering system is disclosed. The smart ordering system may be deployed to order items of any kind. In an example, the smart ordering system is used to order medical supplies such as, e.g., at least medicines, pills, and/or pill bottles. The smart ordering system may analyze history or histories of product orders and related events. As an example, the smart ordering system may predict at least one optimal inventory count of a product, e.g., for near-term needs based on any or any combination or all for the following input variables: minimum stock quantity for the product, recent product usage, seasonal product usage, expiring product stock, and/or past waste events. The smart ordering system may calculate a minimum quantity of product necessary to order. The smart ordering system may compare suppliers for a product based on any or any combination of all of the following input variables: prices and/or price discounts offered, product availability and/or shortages, bulk ordering options for the product, and/or lead time (e.g., length of time between ordering the product and delivery arrival of the product). The smart ordering system may submit ordering information for approval by, e.g., a clinic, pharmacy, hospital, or the like. In some examples the smart ordering system may, if an order for a particular product is approved or times out, submit the order via an API of the supplier (i.e., that hooks into the supplier's software) and/or via a URL/website, e.g., through an automated ordering system. In the alternative the order may be done manually.

In various examples an inventory management system may deploy an anomaly detection process to determine whether changes in unit count are appropriate. A unit count (as a non-limiting example a count of medicine pill in one or more medicine pill bottles) may be updated based at least in part on a change in a weight measurement associated with one or more pads or scales. A “normal” (e.g., expected) change to the unit count may be derived based on, e.g., at least variance calculations, number of standard deviations from the mean, and/or other measures of central tendency. In some examples a range or distribution of “normal” unit count changes may be calculated. If the unit count change is outside the range of “normal” unit count changes (i.e., if the unit count change is an outlier), the system may supply an alert or an alarm to a system user.

In some examples a human user of an inventory management system, such as, e.g., an employee, may be identified and/or authenticated. Such identification and/or authentication may be accomplished by using any, any combination, or all of the following techniques: facial recognition, fingerprint recognition, password login, BLUETOOTH® (including BLE), a smart device carried and/or worn by the person (e.g., at least a smartphone, smart watch, tablet, and/or smart earbuds), tags such as, e.g., at least RFID, NFC, and/or UltraWideband (UWB), and/or a combination of camera (for example, at least one optical camera) recognition and at least one unique marking (as non-limiting examples, a nametag, a badge, or a QR code on a facial or partial facial covering or mask) worn or carried by the person.

In some examples product interaction identification may be enabled in an inventory management system. A product may be identified using any combination or all of the following techniques: Deploying separate pads or scales for each product, affixing labels or tags (such as, e.g., RFID, NFC, or UWB tags) to the product, using a camera in combination with Object Character Recognition (OCR), and/or triangulating with multiple cameras in combination with OCR.

In some examples product identification in an inventory management system may be refined or enhanced by tracking shelf weights over time. As an example, the weight of a shelf or container in a cabinet may be measured, tracking multiple products. Interaction with such products may be tracked over time via smart labels or tags (e.g., RFID, NFC, or UWB) and/or a camera or multiple cameras in conjunction with OCR. A measured weight change may then be used to identify a particular product.

In some examples scales or pads in a cabinet used in an inventory management system may be characterized or graded (e.g., as passing). Raw measurements taken from a scale or pad may be unitless numbers generated by an analog-to-digital converter (A/D) coupled to the scale or pad. A number of zero raw measurements (i.e., measurements taken with no weight on the scale or pad) are taken for a scale or pad. In some examples a number of raw measurements performed may be greater than a threshold value. In an example the threshold value is 500 measurements. In a first test, it is determined whether at least a threshold percentage of the measurements were successful. In some examples the threshold percentage is 80%. Scales or pads that pass the first test may be calibrated to obtain a weight-to-raw ratio for the scale or pad. In a second test, it is determined whether the standard deviation of the measurements is less than a first ceiling value. In an example the first ceiling value is 0.005 grams. In a third test, it is determined whether the mean average measurement error is less than a second ceiling value distant from zero grams. In an example the second ceiling value is 0.05 grams. Scales or pads that pass the three tests may be characterized as passing.

A product setup process in an inventory management system may be performed in some examples. In one example, any, any combination, or all of a picture, barcode, SKU number, user entry of a product name, and/or the like may be used. As an example, a picture of a product or a barcode may be OCR scanned for product identification, dosage, bottle quantity/capacity, and/or the like. A database may be accessed for weight information for the product. If such weight information is not available, a user may be queried to provide manual entry of such weight information and/or walked through setup of product information, e.g., via drop-down menus or the like. In an example, manual input may include any combination or all of the weight of a full bottle of the product (e.g., medicines such as pills or the like), the weight of a pill, and/or the number of pills in a bottle. An example of an automated walkthrough process may include any, any combination, or all of the user placing a full bottle on a scale or pad and recording the weight, placing a predetermined number of pills on the scale or pad and recording the weight, and/or entering the quantity of pills in a full bottle.

In some examples Practice Management Software (PMS) may be integrated with scales or pads in a cabinet in an inventory management system. An order to dispense a specific quantity of a particular product may be generated via PMS by a user, e.g., an employee. The user may update a product unit count on various scales or pads. A unit count change may be compared with the quantity specified by the PMS, and an alert or an alarm may be generated in the event of a mismatch. In addition or in the alternative, the identity of the user may be compared with the identity of an employee who has been assigned to dispense the product in question. In the event the identities do not match, an alert or an alarm may be generated by the system.

In various examples, scale or pad measurement (per weight read) may be refined or enhanced to improve sensitivity or accuracy. Multiple raw weight measurements may be acquired at a first sample rate. In an example the first sample rate is a relatively high sample rate with respect to a second sample rate. The signal may in some examples be filtered to remove outlier measurements, for example, measurements that fall more than a threshold distance or number of standard deviations from the mean or deviations from the median. An average of the raw weight measurements may be calculated to acquire a single sample at a desired sample rate. In some examples the desired sample rate is the second sample rate, which is a relatively low sample rate with respect to the first sample rate. The sample may be converted to a “real-world” weight measurement (e.g., a weight measurement in grams) by calibrating the scale or pad, for example by applying historical ratio information of raw weight (unitless A/D output from a scale or pad) to grams stored by the system. The “real-world” weight measurement may be refined by combination with weight measurements from other scales or pads tracking the same product.

In some examples weight changes per scale or pad may be used to enhance or improve scale sensitivity or accuracy. A running history of weight samples may be maintained. In one aspect the sample history may be filtered or smoothed to obtain cleaner, or more “normal” data. Steady state periods may be defined for the sample data, including start and end times for such periods, during which periods the weight measurements change by less than a predetermined ceiling value. Relative weight change events may be computed as differences between weight measurements taken at an end of one steady state period and weight measurements taken at the start of the next such period. Employing such a technique may account for short-term drift of the scale or pad during the steady state period. Weight change events that exceed a predetermined threshold value may be recorded or published.

In some examples a unit count calculation process may be employed. The unit count calculation process may take as inputs at least one of the scale container weight (e.g., the weight of an empty bin, in which medicine or pill bottles may ordinarily be stored, on a scale or pad), product container weight (e.g., the weight of a medicine or pill bottle), full product container count (e.g., the number of pills in a full bottle), and/or product unit weight (e.g., the weight of an individual pill). The weight measurement may then be offset to account for the scale container weight. An error check may then be performed by comparing the measured weight to the scale container weight. If the measured weight is greater than zero grams but less than the scale container weight, the system may generate an alert or an alarm. The remaining weight measurement value, after offsetting to account for the scale container weight, may be rounded down based on the number of full containers on the scale or pad, the weight of a full container, and/or the unit weight of an individual pill given the known total number of pills per container. The weight measurement may then be offset to account for the product container weight. An error check may then be performed by comparing the measured weight to the product container weight. If the measured weight is greater than zero grams but less than the product container weight, the system may generate an alert or an alarm. The remaining weight (after offsetting for the product container weight) may be used to calculate a partially filled unit (e.g., pill) count.

A bin may contain a number of pill bottles with one of the bottles being partially full and the remaining pill bottles being full. As a non-limiting example, if a bin containing three full bottles and a fourth, partially full bottle is weighed, the number of pills in the partially full bottle may be determined by subtracting from the measured weight on the scale or pad the weight of an empty bin, three times the weight of one full bottle, and the weight of one empty bottle, and then dividing the resulting weight measurement by the weight of an individual pill. The total number of pills in the bin may be calculated by adding three times the number of pills in a full bottle to the number of pills in the partially filled bottle.

An error check may be performed by determining whether excessive rounding has occurred for the unit count calculation (for example, if the number of units calculated is at least a threshold fraction more or less than an integer), and the system may generate an alert or an alarm if such is called for based on the rounding determination. A change in unit count when compared with the previous calculation may be computed. The system may analyze whether the change in unit count is unusual or anomalous when compared to historical unit count changes, and generate an alert or an alarm if the check is found to be true (as a non-limiting example, if the unit count change exceeds an average of a predetermined number of historical unit count changes by more than a predetermined threshold value).

In some examples smart events logic may be applied to an inventory management system to refine inventory tracking by unit weight and count. An auto-zero value may be determined by removing approximately all weight from a scale or pad. Approximately all weight may be removed from the scale or pad except for a container, e.g., a medicine pill bottle, on the scale or pad, yielding an auto-zero value with only container weight and a zero unit count. If weight is added to the scale or pad after removal of weight from the scale or pad, such weight change events may be combined to enhance system knowledge. In an inventory restocking event, weight may be added, indicating that a product was added to the scale or pad. A known value for full product container count may be used to add stock to the scale or pad. A change in unit count may be determined by using the difference between a full product container weight and a new weight measurement for the scale or pad.

In various examples a scale or pad may be calibrated by performing raw scale weight measurements using items with known weights. In some examples multiple items each having different weights may be used. In one example three such items may be used. For each known weight, a weight-to-raw ratio may be calculated. As a non-limiting example, a raw (e.g., unitless) output of an A/D coupled to the scale may be the number 5,000, and may correspond to one gram in “real world” weight. The ratios may be averaged to obtain a ratio for the calibrated scale or pad. Such a ratio may then be used by the system in subsequent scale events (obtaining weights of items on the scale or pad).

In an example, identity of a human user, e.g., an employee, may be combined with data from scales or pads to enhance system security and/or accuracy by reading measurements from the scales or pads at various times and assigning a most recently identified employee to conduct scale events such as, e.g., weighing items. In another example, identity of a human user, e.g., an employee, may be combined with data from scales or pads and usage of a door to a cabinet housing the scales or pads to enhance system security and/or accuracy. If the door is open and/or a particular employee has been recently identified, weight measurements may be taken from scales or pads, and the most recently identified employee may be assigned to conduct scale events such as, e.g., weighing items. In another example, identity of a human user, e.g., an employee, may be combined with data from scales or pads and usage of a lockable door to a cabinet housing the scales or pads to enhance system security and/or accuracy. If an employee is identified or authenticated, the system may unlock the door. The system may lock the door after the door is closed, and/or after the door is closed and remains closed for predefined threshold amount of time. If the door is open and/or if an employee has been identified recently, weight measurements may be taken from scales or pads, and the most recently identified employee may be assigned to conduct scale events such as, e.g., weighing items.

In some examples a system routine self-analysis may be performed. All local services may be restarted, and all active scales or pads may then be identified and labeled or otherwise characterized to ensure accurate measurement functionality for the system. Wired or wireless connection to the Internet or cloud may be tested. Communication with various backend servers, networks, and/or databases may be tested. Upon failure of any such connection during error-checking testing, the system may generate an alert or an alarm.

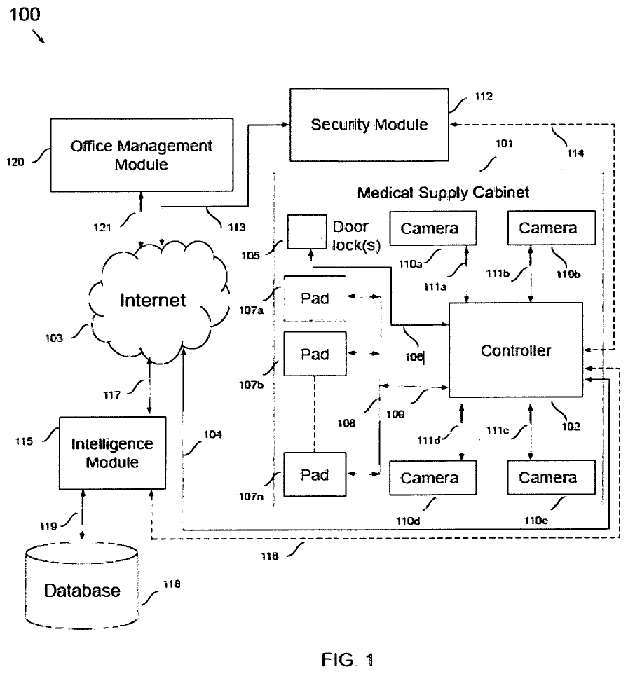

FIG. 1 is a block diagram of an inventory management system architecture 100 in accordance with various aspects of the subject disclosure. With reference to FIG. 1, in one or more examples, the system 100 can comprise at least one medical supply cabinet 101, which may contain inventory items such as, for example, drugs or pill bottles or other medical supplies. The cabinet 101 can include a controller 102. In various examples the controller 102 can be either integral to the cabinet 101 or reside external to the cabinet 101. The controller 102 can be any of, for example, a processor, a computer, a microcontroller, a state machine, an application specific integrated circuit (ASIC), or any other type of controller. In some examples the controller 102 may be implemented as multiple, distinct control units. The controller 102 can be coupled to a cloud-based network such as the Internet 103 by connection 104, which may in various aspects be a wired bus or any of various wireless connections.

In various examples, the medical supply cabinet 101 may, but need not, include at least one door (not shown) and at least one door lock 105 that is coupled to the controller 102 by a connection 106, which may in various aspects be a wired bus or any of various wireless connections, such that absent a signal from the controller 102, a door or doors will remain in locked states. In an example the controller 102 may not send a signal to the at least one door lock 105 to open a door unless a user attempting to access the cabinet 101 has been identified and/or authenticated. In an example the controller 102 may send an alert message to an office manager or system administrator if a user attempting to access the cabinet 101 has not been identified and/or authenticated. In an example, if a door is opened and then closed again for a predefined length of time, for a non-limiting example four seconds, the controller 102 may send a signal to the at least one door lock 105 to lock the door. The cabinet 101 also includes pads 107a-107n. While three pads 107a-107n are shown for purposes of simplicity, it would be readily appreciated that there could be any number of pads 107a-107n in the cabinet 101. The pads 107a-107n can be suitable for inventory items to reside thereon, and can comprise scales to measure the respective weights of such inventory items. Each pad 107 may comprise a scale or may include a scale embedded in or integral to the pad 107. The pads 107a-107n can be coupled to each other and to the controller 102 by connections 108, 109, which can be wired buses or any of various wireless connections. Any information the pads 107a-107n detect, such as, for example, weight information about one or more inventory items placed on or removed from a pad 107, can be retrieved by and/or controlled to be received by the controller 101 via connections 108, 109.

In various examples, the medical supply cabinet 101 may also include at least one camera 110a-110d. While four cameras 110a-110d are shown for purposes of simplicity, it would be readily appreciated that there could be any number of cameras 110a-110d in the cabinet 101. In various aspects the cameras 110a-110d may, for example, be optical cameras, ultrasound cameras, ultrasonic cameras, infrared cameras, or other of various types of cameras. In some examples, the cameras 110a-110d can be coupled to the controller 102 by, respectively, connections 111a-111d. It would be understood by those of skill that the connections 111a-111d can be wired buses or any of various wireless connections. In an example, the cameras 110a-110d can detect whether an inventory item is placed on an incorrect pad 107a-107n. In an example the cameras 110a-110d can read inventory product labels in the medical supply cabinet 101 and/or scan bar code information on inventory products in the cabinet 101 for product information. Any information the cameras 110a-110d detect can be retrieved by and/or controlled to be received by the controller 101 via connections 111a-111d.

The system 100 may, in various examples, include a security module 112, such as, by way of non-limiting example, a processor, which can be coupled to the network-based cloud or the Internet 103 by connection 113. The security module 112 may in addition or in the alternative be coupled directly to the controller 102 by a connection 114 (shown in dashed lines). The connections 113, 114 may be wired buses or any of various wireless connections. In various examples, the security module 112 may identify and/or authenticate a user of the system 100, such as, for example, an employee. The security module 112 may identify and/or authenticate the user by any of a number of non-limiting techniques such as, for example, RFID card, facial recognition, voice recognition, fingerprint recognition, iris scan, passcode recognition, gesture recognition, keypad entry (for example, on a tablet such as an iPad® or a tablet running the Android® operating system), wireless proximity detection technologies such as BLUETOOTH®, BLUETOOTH® beacons, or NFC, longer range wireless technologies such wireless LAN, e.g., 802.11 standards, and WAN or cellular, including 2G, 3G, 4G, or 5G technologies, and/or a wearable device such as, for example, an Apple Watch® or a watch running the Android® operating system.

The system 100 may, in various examples, include an intelligence module 115, such as, by way of non-limiting example, a processor, which may be coupled to the network-based cloud or the Internet 103 by a connection 117. In another aspect, the intelligence module 115 may in addition or in the alternative be coupled directly to the controller 102 by a connection 116 (shown in dashed lines). The connections 116, 117 may be wired buses or any of various wireless connections. In other examples the intelligence module 115 may be integral to or embedded in or running on the controller 102. In an example, the intelligence module 115 may be coupled to a database 118 by a connection 119. The connection 119 may be a wired bus or any of various wireless connections. In some examples the intelligence module 115 may comprise a software module running on or controlled by a computing device such as the controller 102. The intelligence module 115 may be implemented in any of various non-limiting ways such as, e.g., software or firmware. The intelligence module 115 may in some embodiments comprise an artificial intelligence engine or processor that can operate in response to commands from the controller 102, for example based on information the controller 102 has obtained from at least one pad 107a-107n (e.g., scale weight of an inventory item placed on or removed from the pad 107) and/or at least one camera 110a-110d via, respectively, connections 108, 109, 111a-111d, to place orders for new inventory items.

In an example the intelligence module 115 may help the inventory management system 100 compensate for possible power outages. If power were lost, the system 100 could reset and the pads 107a-107n could consequently have their scales reset to zero weight values while there were inventory items on the pads 107a-107n, thereby precluding the system 100 from knowing how many items were located on each pad 107a-107n. It would not be practical to revert to a system 100 setup mode, remove all of the inventory items from the cabinet 101, and reset the scale weight values to zero for all the pads 107a-107n. To alleviate this concern, the intelligence module 115 can monitor prescriptions written by doctors for a given number of inventory items and learn how many grams of weight are removed, and from which pad 107 such grams of weight are removed, and the type of inventory item that is placed on that specific pad 107, and ultimately how much each inventory item weighs. In an example the intelligence module 115 may compare reduced weights on each pad 107a-107n in the cabinet 101 to the quantity of the medication prescribed on the prescription and thus learn the weight of each item, pill, or volume (e.g., of vaccine in a vial) over time. In an aspect, the intelligence module 115 can also determine how many inventory items or partial inventory items (e.g., pills from a bottle) were removed because the intelligence module 115 has learned the weight of each inventory item and/or partial inventory item (as a non-limiting example, each pill in a pill bottle). By way of non-limiting example, if a pad 107 scale weight reading is reduced by 2.5 grams and the associated prescription requires 5 pills, the intelligence module 115 would determine that the weight of each pill is 0.5 grams. And, also as a non-limiting example, if the weight of each pill is 0.5 grams and a weight value is reduced by 2.5 grams, the intelligence module 115 would determine that 5 pills had been removed. In an example, at initial calibration upon first startup of the system 100, the system 100 may be calibrated by a user placing, for example, on a pad 107a-107n having zero weight value, a bottle of pills and entering the number of pills in the bottle, then removing the bottle and placing one pill on the pad 107a-107n, enabling the system 100 to record the weight of each pill. The user may in another example place a vial of vaccine on the pad 107a-107n, enter the number of cubic centimeters (cc) (it should be appreciated that any volume measurement may be used) of vaccine that is in the vial, and remove one cc of vaccine from the vial, thereby enabling the system 100 to determine the weight of one cc of vaccine by subtracting the new weight value from the previous weight value. In an example the intelligence module 115 may use stored prescription information for patients to send notifications to the patients, e.g., by way of text, email, and/or phone call, to remind the patients that it is time to take medicine and/or to order more medicine.

In various examples the database 118 may contain inventory product quantities and/or counts. In other aspects the database 118 may also contain information about any or all of the following: available supplies of various medical products, product expirations, the existence of any epidemics of illness related to any medical products being stored in the medical supply cabinet 101, price reductions or promotions being offered or advertised to be coming by any suppliers of medical products, prior history of quantities used of medical products, and/or any seasonal factors that may impact supply or demand of such medical products.

In some examples the system 100 may optionally include an office management module 120, such as, by way of non-limiting example, a processor. The office management module 120 can be coupled to the network-based cloud or the Internet 103 by connection 121, which may be a wired bus or any of various wireless connections. In some examples the office management module 120 may comprise a software module running on or controlled by a computing device or processor. The office management module 120 may be implemented in any of various non-limiting ways such as, e.g., software or firmware. The office management module 120 may be software supplied by a third-party vendor. In some examples the intelligence module 115 may direct, by way of the network-based cloud or the Internet 103 via connections 117, 121, the office management module 120 to procure some amount of at least one inventory item that the intelligence module 115 has determined needs to be ordered. In other examples the intelligence module 115 may place such an order directly through a web portal of the vendor via a connection 117 and the network-based cloud or the Internet 103.

In some examples any, any combination of, or all of the controller 102, security module 112, intelligence module 115, and/or office management module 120 may reside in the network-based cloud or the Internet 103. In some examples any, any combination of, or all of the controller 102, security module 112, intelligence module 115, and/or office management module 120 may be implemented as at least one processor. In some examples any, any combination of, or all of the controller 102, security module 112, intelligence module 115, and/or office management module 120 may be implemented as the same processor.

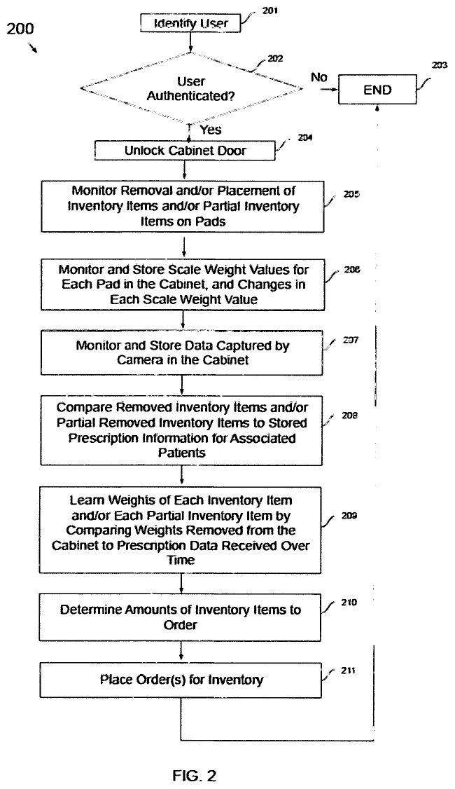

In operation in accordance with various aspects, the system 100 may follow some or all of the flow and actions depicted in FIG. 2, in which a non-limiting example of a flow chart 200 depicting flow and steps in accordance with various aspects and embodiments of the subject disclosure is illustrated. It should be readily understood that the actions need not necessarily be performed in the order shown, or in any particular order. It should also be understood that not all of the depicted actions need be performed, and that some actions may be either optionally performed or omitted.

With reference to FIG. 2, in action 201 a user of the inventory management system 100 is identified. The user may be an employee attempting to gain access to the medical supply cabinet 101. The user may be identified by any of a number of non-limiting techniques such as, for example, RFID card, facial recognition, voice recognition, fingerprint recognition, iris scan, passcode recognition, gesture recognition, keypad entry (for example, on a tablet such as an iPad® or a tablet running the Android® operating system), wireless proximity detection technologies such as BLUETOOTH®, BLUETOOTH® beacons, or NFC, longer range wireless technologies such as wireless LAN, e.g., 802.11 standards, and WAN or cellular, including 2G, 3G, 4G, or 5G technologies, and/or a wearable device such as, for example, an Apple Watch® or a watch running the Android® operating system. Control flow then proceeds to action 202.

In action 202 it is determined whether the user is authenticated. If the user is not an authorized user of the system 100, control flow proceeds to end at action 203, namely, the door(s) is maintained in its locked state and the user is not permitted access to the medical supply cabinet 101, and, in some aspects (not shown), an alert message may be sent to an office manager or system administrator. If in action 202 it is determined that the user is an authorized user of the system 100, control flow proceeds to action 204. In action 204 the door(s) to the medical supply cabinet 101 is unlocked to allow entry to the medical supply cabinet 101 by the user. In other examples a door lock mechanism may not be needed to prevent or grant entry by the user to the medical supply cabinet 101. Control flow then proceeds to action 205.

In action 205 the removal and/or placement of inventory items and/or partial inventory items on pads 107a-107n by the user is monitored. Control flow then proceeds to action 206. In action 206 the scale weight values for each pad 107a-107n in the medical supply cabinet 101, and the changes in scale weight values for each pad 107a-107n in the cabinet 101, are monitored and stored. This enables the system 100 to determine which inventory item has been removed and the quantity of that item that was removed. If an inventory item were misplaced after removal, i.e., on an incorrect pad 107a-107n, an error would be detected since the weight value on the incorrect pad 107a-107n would have changed by an unexpected amount. The system 100 could then alert the user to place the item on the correct pad 107a-107n. Control flow then, optionally, proceeds to action 207.

In an example, in action 207, data captured by any or all of the cameras 110a-110d in the medical supply cabinet 101 may optionally be monitored and stored. The system 100 may use this data to detect if an inventory item has been misplaced on an incorrect pad 107a-107n, or the system 100 may read product labels and/or scan bar codes from the data. In some aspects (not shown), if the door is opened and then closed again for a predefined length of time, by way of non-limiting example four seconds, the controller 102 may lock the door. Control flow then proceeds to action 208.

In action 208, in an example, removed inventory items and/or partially removed inventory items from the medical supply cabinet 101, by way of non-limiting example pills from bottles or vials from vaccine containers, can be compared to stored prescription information for patients associated with the inventory item(s) in order to eliminate errors. In some aspects (not shown), the stored prescription information may be used to send a notification to a patient, e.g., by way of text, email, and/or phone call, to remind the patient that it is time to take medicine and/or to order more medicine. Control flow then, optionally, proceeds to action 209.

In an example, in action 209, the weights of each inventory item and/or each partial inventory item from the medical supply cabinet 101, by way of non-limiting example pills from bottles or vials from vaccine containers, can be learned over time by the system 100 (e.g., via the intelligence module 115) by comparing weights of removed or partially removed inventory items to prescription information associated with patients who are to receive those inventory items. By way of non-limiting example, if a scale weight reading is reduced by 2.5 grams and the associated prescription requires 5 pills, the software would determine that the weight of each pill is 0.5 grams. Conversely, and also as a non-limiting example, if the weight of each pill is 0.5 grams and a weight value is reduced by 2.5 grams, the intelligence module 115 would determine that 5 pills had been removed. Control flow then proceeds to action 210.

In action 210 the system 100 (e.g., via the intelligence module 115) determines an amount or amounts of one or more inventory items to be ordered. In various examples the determination is made according to any one or more of the following: seasonal factors, past usage or order history, price promotions from different suppliers or vendors, supply shortages, extant epidemics, and/or product life or expiration date. Control flow then proceeds to action 211.

In action 211 an order for at least one quantity of at least one inventory item is placed. The order may be placed by the third-party office management module 120 by way of the network-based cloud or the Internet 103 or directly through a web portal of the vendor via the network-based cloud or the Internet 103.

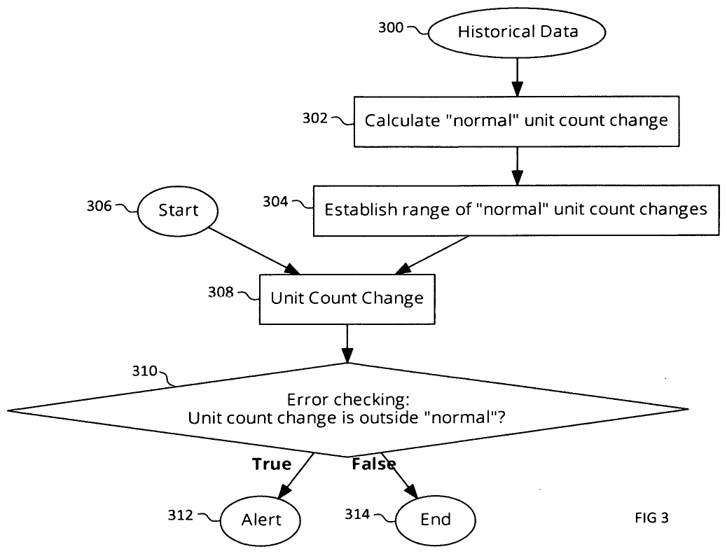

In the flow chart shown in FIG. 3, a non-limiting example of an anomaly detection process in accordance with various aspects of the subject disclosure is depicted. The actions depicted may be performed by at least one processor in an inventory management system. With reference to FIG. 3, historical data may be generated and used in action 302 to calculate a “normal” unit count change to inventory, e.g., medicine pills. For example, data may be stored by the system that indicates the value of a typical or “normal” amount of pills ordinarily taken from a pill bottle for a particular product. Control flow then proceeds to action 304, in which a range of “normal” unit count changes may be established. Routine 306 may be started to cause, in action 308, a unit count change to be determined. Control flow then proceeds to action 310, in which error checking is performed and it is determined whether the unit count change falls outside of a “normal” range. If the unit count change is determined to fall outside of a “normal” range, control flow proceeds to action 312, in which an alert or an alarm is generated. If, in action 310, it is determined that the unit count change falls within a “normal” range, control flow proceeds to action 314 and the anomaly detection process ends.

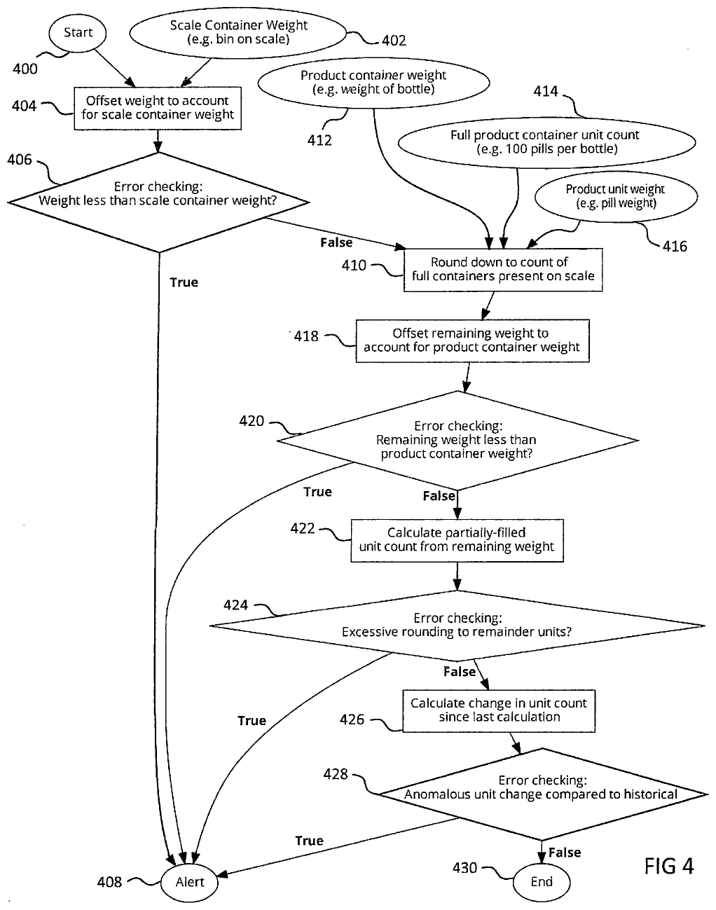

In the flow chart shown in FIG. 4, a non-limiting example of a unit count calculation process in accordance with various aspects of the subject disclosure is depicted. The actions depicted may be performed by at least one processor in an inventory management system. With reference to FIG. 4, a unit count process begins in action 400. Scale container weight for a pad or scale in a cabinet (as a non-limiting example an empty bin that would ordinarily carry pill bottles on a scale or pad) may be obtained in action 402. Control flows proceeds to action 404, in which a weight measurement from the scale or pad may be offset to account for the scale container weight. Control flow proceeds to action 406, in which error checking may be performed to determine whether the weight measurement from the scale or pad is less than the scale container weight. If the weight measurement from the pad or scale is less than the scale container weight measurement, control flow proceeds to action 408, in which an alert or an alarm may be generated. If the weight measurement from the pad or scale is not less than the scale container weight measurement, control flow proceeds to action 410. A product container weight (as a non-limiting example, a weight of a pill bottle) may be obtained in action 412, a full product container unit count (as a non-limiting example, a total number of pills (perhaps, e.g., 100 pills) in a pill bottle) may be obtained in action 414, and a product unit weight (as a non-limiting example, a weight of an individual pill from a pill bottle) may be obtained in action 416. The product container weight, the full product container unit count, and the product unit weight may be used in action 410 to round the weight measurement down to a count of full product containers present on the scale or pad. Control flow then proceeds to action 418.

With continued reference to FIG. 4, in action 418 the weight measurement resulting from action 410 may be offset to account for the product container weight. Control flow then proceeds to action 420, in which error checking may be performed to determine whether the weight value resulting from action 418 is less than the product container weight. If the weight value is less than the product container weight, control flow proceeds to action 408, in which an alert or an alarm may be generated. If the weight value is not less than the product container weight, control flow proceeds to action 422, in which a partially filled unit count (e.g., in the event one or more of the bottles is not full (i.e., has had a number of pills removed from it)) may be calculated from the weight value resulting from action 420. In an example, a bin may contain a number of pill bottles with one of the bottles being partially full and the remaining pill bottles being full. As a non-limiting example, if a bin containing three full bottles and a fourth, partially full bottle is weighed, the number of pills in the partially full bottle may be determined by subtracting from the measured weight on the scale or pad the weight of an empty bin, three times the weight of one full bottle, and the weight of one empty bottle, and then dividing the resulting weight measurement by the weight of an individual pill. Control flow then proceeds to action 424, in which error checking may be performed to determine whether excessive rounding to remainder units has occurred (for example, if the number of units calculated is at least a threshold fraction more or less than an integer). If excessive rounding to remainder units has occurred, control flow proceeds to action 408, in which an alert or an alarm may be generated. If excessive rounding to remainder units has not occurred, control flow proceeds to action 426, in which a change in the unit count with respect to a previous unit count may be calculated. Control then proceeds to action 428, in which error checking may be performed to determine whether the calculated unit count change is anomalous when compared with historical unit count data, for example, whether a present unit count change exceeds an average or other measure associated with one or more historical unit count changes by, or by more than, a predefined threshold amount. If a present unit count change is determined to be anomalous when compared with historical unit count data, control flow proceeds to action 408, in which an alert or an alarm may be generated. If a present unit count change is determined not to be anomalous when compared with historical unit count data, control flow proceeds to action 430 and the unit count calculation process ends.

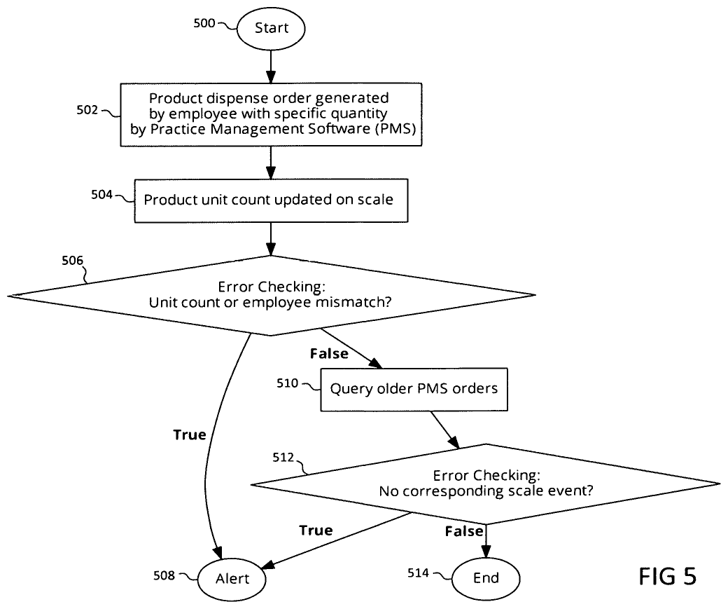

In the flow chart shown in FIG. 5, a non-limiting example of a process for integrating PMS with scales in accordance with various aspects of the subject disclosure is depicted. The actions depicted may be performed by at least one processor in an inventory management system. With reference to FIG. 5, a process for integrating PMS with scales begins in action 500. Control flow proceeds to action 502, in which an order to dispense a product may be generated by a system user such as, e.g., an employee. The order may specify a quantity of items and may be generated through PMS. The PMS may comprise software provided in any form by a third party supplier. The system may include at least one API, that may be provided by the third party supplier, to access the PMS. Control flow proceeds to action 504, in which a product unit count may be updated on a scale or pad in a storage unit such as, e.g., a cabinet. Control flow then proceeds to action 506, in which error checking may be performed to determine whether there is a mismatch between either or both of the updated unit count and the unit count entered in the order, and/or the identity of the system user who removed the pills and the identity of the system user who entered to order. If a mismatch is determined to exist, control flow proceeds to action 508, in which an alert or an alarm may be generated. If a mismatch is determined not to exist, control flow proceeds to action 510, in which one or more historical PMS orders may be queried. Control flow then proceeds to action 512, in which error checking may be performed to determine whether no corresponding scale or pad event has occurred (as a non-limiting example, whether no items have been weighed on a particular pad or scale such that, e.g., no pills have been removed). If it is determined that no corresponding scale or pad event has occurred, control flow proceeds to action 508, in which an alert or an alarm may be generated. If it is determined that a corresponding scale or pad event has occurred, control flow proceeds to action 514 and the process for integrating PMS with scales ends.

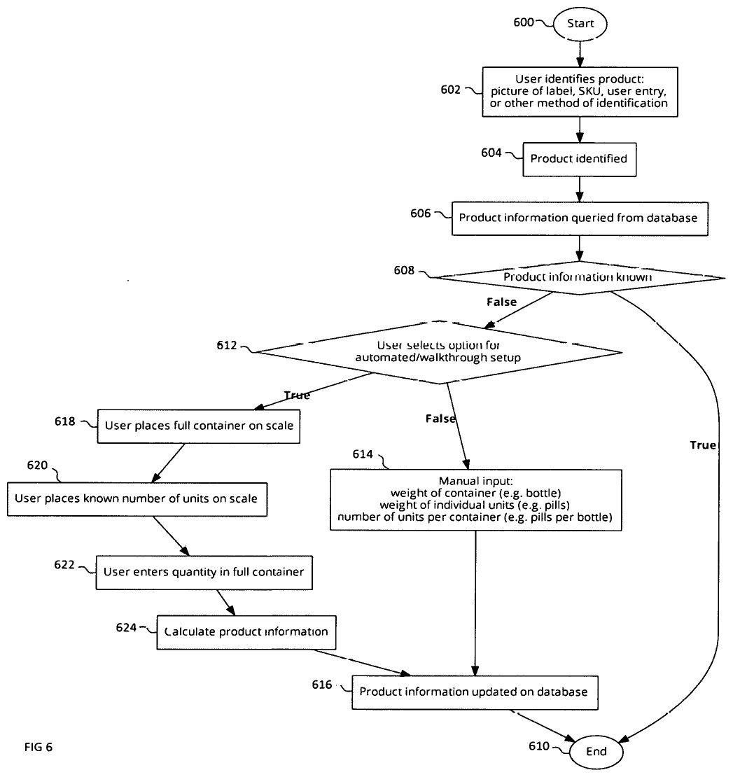

In the flow chart shown in FIG. 6, a non-limiting example of a product setup process in accordance with various aspects of the subject disclosure is depicted. The actions depicted may be performed by at least one processor in an inventory management system. With reference to FIG. 6, a product setup process begins in action 600. Control flow proceeds to action 602, in which a system user may identify a particular product using any of various identification techniques including but not limited to showing a picture of a label, entering an SKU number or other identifying number, scanning a bar code or RFID tag on a smart label, etc. Control flow proceeds to action 604, in which the product may be identified based at least in part on the actions taken in action 604. Control flow proceeds to action 606, in which the system may query a database or other datastore for information about the product, such as, e.g., the count of pills per bottle and/or the weight of an individual pill and/or the weight of an empty bottle. Control flow then proceeds to action 608, in which it may be determined whether the product information is known by the system. If the product information is known by the system, control flow proceeds to action 610 and the product setup process ends.

With continued reference to FIG. 6, if in action 608 the product information is not known by the system, control flow proceeds to action 612. In action 612 a system user may decide whether to select an option to conduct an automated/walkthrough setup for the product. If the user decides not to select an option to conduct an automated/walkthrough setup for the product, control flow proceeds to action 614, in which a user may input manually a weight of a container of the product, e.g., a bottle, a weight of an individual unit, e.g., a pill, and a number of units in the container, e.g., pills per bottle. Control flow then proceeds to action 616, in which information about the product may be updated in a database. Control flow then proceeds to action 610 and the product setup process ends.

With continued reference to FIG. 6, if in action 612 a user decides to select an option to conduct an automated/walkthrough setup for the product, control flow proceeds to action 618, in which a user may place a full container of the product on a scale or pad. Control then proceeds to action 620, in which a user may place a known number of units, e.g., pills, on the scale or pad. Control flow then proceeds to action 622, in which a user may enter a quantity of units, e.g., pills, in a full container, e.g., medicine pill bottle. Control flow then proceeds to action 624, in which product information is calculated. Control flow then proceeds to action 616, in which information about the product may be updated in a database. Control flow then proceeds to action 610 and the product setup process ends.

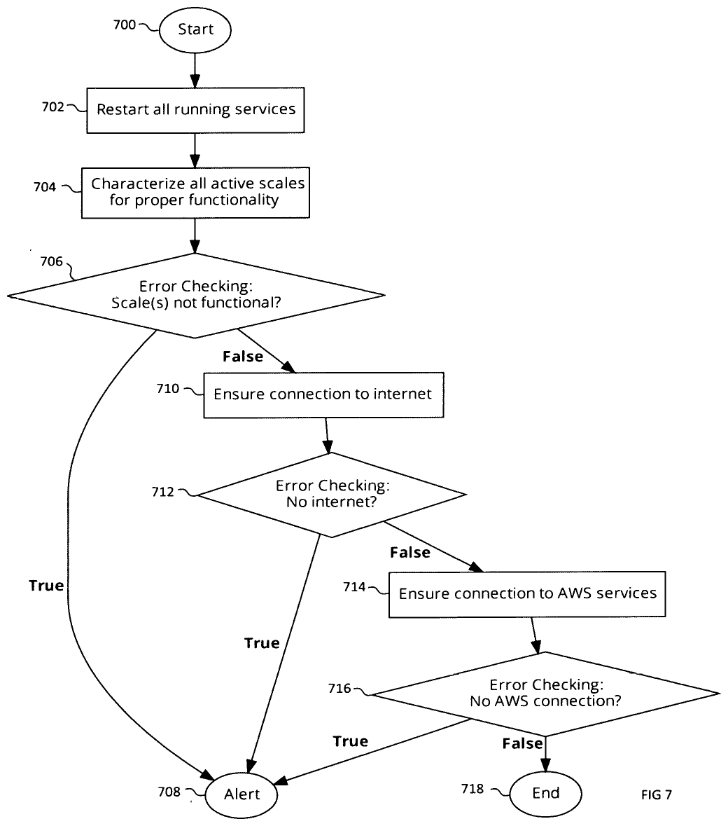

In the flow chart shown in FIG. 7, a non-limiting example of a system routine self-analysis process in accordance with various aspects of the subject disclosure is depicted. The actions depicted may be performed by at least one processor in an inventory management system. With reference to FIG. 7, a system routine self-analysis process begins in action 700. Control flow proceeds to action 702, in which the system may restart all currently running services. Control flow then proceeds to action 704, in which active scales or pads, in some instances all active scales or pads, may be identified and labeled or otherwise characterized to ensure accurate measurement functionality for the system. Control flow then proceeds to action 706, in which error checking may be performed to determine whether at least one scale or pad is functioning accurately. If it is determined that the at least one scale or pad is not functioning accurately, control flow proceeds to action 708, in which the system may generate an alert or an alarm. If in action 706 it is determined that the at least one scale or pad is functioning accurately, control flow proceeds to action 710.

With continued reference to FIG. 7, in action 710, at least one wired or wireless connection of the system, or various components and/or processors thereof, to the Internet or cloud may be tested. Control flow proceeds to action 712, in which error checking may be performed to determine whether such connection to the Internet or cloud exists. If no such connection to the Internet or cloud can be established, control flow proceeds to action 708, in which the system may generate an alert or an alarm. If in action 712 it is determined that such connection with the Internet or cloud exists, control flow proceeds to action 714, in which at least one wired or wireless connection of the system, or various components and/or processors thereof, to any of various backend servers, networks, and/or databases may be tested, including, e.g., but not limited to Amazon Web Service (AWS). Control flow proceeds to action 718, in which error checking may be performed to determine whether such connection to, e.g., AWS, exists. If no such connection can be verified, control flow proceeds to action 708, in which the system may generate an alert or an alarm. If in action 718 such connection to, e.g., AWS, is verified, control flow proceeds to action 718 and the system routine self-analysis process ends.

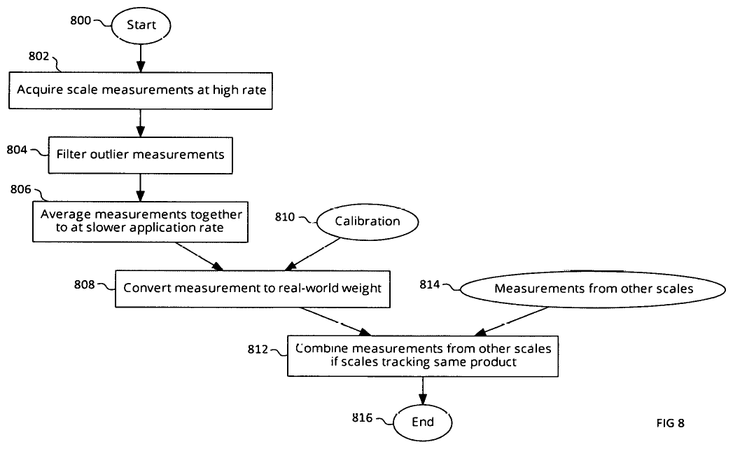

In the flow chart shown in FIG. 8, a non-limiting example of a scale measurement process in accordance with various aspects of the subject disclosure is depicted. The actions depicted may be performed by at least one processor in an inventory management system. With reference to FIG. 8, a scale measurement process begins in action 800. Control flow proceeds to action 802, in which various raw weight measurements for a scale or pad (e.g., digital representations of analog values output by an A/D coupled to the scale or pad) may be acquired at a first predetermined sample rate. In one example the first predetermined sample rate is 10 Hz. Control flow then proceeds to action 804, in which outlier measurements (as a non-limiting example, measurements that fall more than a threshold distance or number of standard deviations from the mean or deviations from the median), may be removed by, e.g., filtering. Control flow then proceeds to action 806, in which raw weight measurements for the scale or pad may be averaged together at a second predetermined sample rate that may be relatively lower than the first predetermined sample rate. In one example the second predetermined sample rate is 1 Hz. Control flow proceeds to action 808, in which a weight measurement for the scale or pad may be converted to a “real-world” weight measurement (e.g., a weight measurement in grams) by calibrating the scale or pad, for example by applying historical ratio information of raw weight (unitless A/D output from a scale or pad) to grams stored by the system (action 810). Control flow proceeds to action 812, in which the “real-world” weight measurement may be refined by combining the “real-world” weight measurement with weight measurements from other scales or pads tracking the same product (action 814). Control flow then proceeds to action 816, in which the scale measurement process ends.

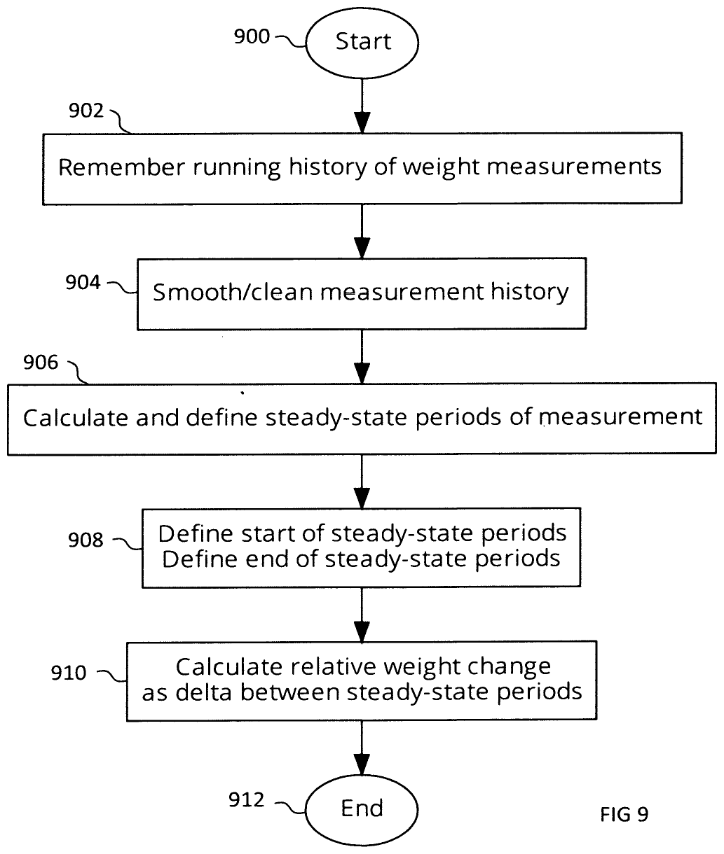

In the flow chart shown in FIG. 9, a non-limiting example of a scale weight change process in accordance with various aspects of the subject disclosure is depicted. The actions depicted may be performed by at least one processor in an inventory management system. With reference to FIG. 9, a scale weight change process begins in action 900. Control flow proceeds to action 902, in which a running history of weight measurements may be maintained. Control flow then proceeds to action 904, in which a running history of weight measurements may be filtered or smoothed to obtain cleaner data. Control flow then proceeds to action 906, in which at least one steady state period of measurement may be calculated and defined, during which period the weight measurements change by less than a predetermined ceiling value. Control flow then proceeds to action 908, in which at least one start time and at least one end time may be defined for the at least one steady state period. Control flow then proceeds to action 910, in which at least one relative weight change may be calculated as a difference between at least one weight measurement taken at an end of one steady state period and at least one weight measurement taken at a start of a next steady state period. Control flow then proceeds to action 912, in which the scale weight change process ends.

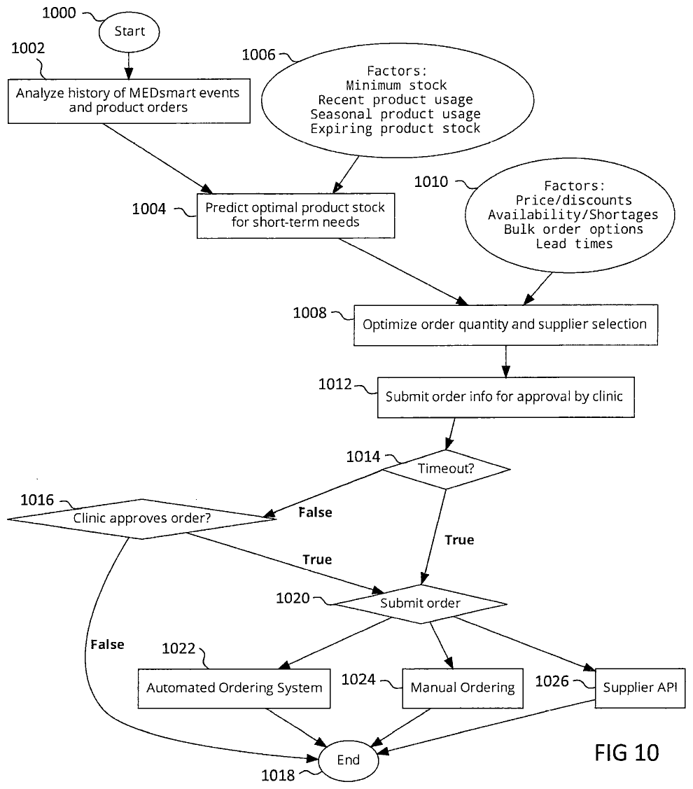

In the flow chart shown in FIG. 10, a non-limiting example of a smart ordering system process in accordance with various aspects of the subject disclosure is depicted. The actions depicted may be performed by at least one processor in an inventory management system. With reference to FIG. 10, a smart ordering system process begins in action 1000. Control flow proceeds to action 1002, in which the system may analyze a history or histories of product orders and related events. Control flow then proceeds to action 1004, in which the system may predict at least one optimal inventory or stock count for a product, e.g., for near-term needs based on any or any combination or all for the following input variables: minimum stock quantity for the product, recent product usage, seasonal product usage, and/or expiring product stock (action 1006). Control flow proceeds to action 1008, in which the system may optimize supplier selection for the product and optimize at least one order quantity for the product (e.g., calculate a minimum quantity of the product necessary to order) by comparing suppliers based on any or any combination of the following input variables: prices and/or price discounts offered, product availability and/or shortages, bulk ordering options for the product, and/or lead time (e.g., length of time between ordering the product and delivery arrival of the product) (action 1010). Control flow proceeds to action 1012, in which the system may submit ordering information for approval by, e.g., a clinic, pharmacy, hospital, or the like. Control flow then proceeds to action 1014, in which it may be determined whether a timeout has occurred, e.g., whether a threshold amount of time since the ordering information was submitted has expired without the clinic or the like approving the order. In one example the timeout period is one day. If a timeout has not occurred, control flow proceeds to action 1016, in which the system determines whether the clinic or the like has approved the order. If the order has not been approved, control flow proceeds to action 1018 and the smart ordering system process ends.

With continued reference to FIG. 10, if in action 1016 it is determined that the order has been approved, or if in action 1014 it is determined that a timeout has occurred, control flow proceeds to action 1020. In action 1020 the system determines one of three potential ways to submit an order for the product, and control flow proceeds accordingly. The system may, in action 1022, submit the order for the product using an automated ordering system such as, e.g., a Web browser-based system. In addition or in the alternative, the order may, in action 1024, be submitted manually by a system user. In addition or in the alternative, the system may, in action 1026, submit the order for the product via an API of the supplier (i.e., an API that hooks into the supplier's software). Once the order for the product has been submitted, control flow proceeds to action 1018 and the smart ordering system process ends.

In non-limiting examples, a computing system is provided, the system comprising one or more processors and one or more memories that stores executable instructions that, when executed by the one or more processors, can facilitate performance of the operations as described herein, including the non-limiting methods as illustrated in the various flow diagrams of the drawings.

Information and signals may be represented using any of a variety of different techniques. For example, data, instructions, commands, information, signals, bits, or symbols that may be referenced throughout the description may be represented by voltages, currents, electromagnetic waves, magnetic fields or particles, optical fields or particles, ultrasonic waves, projected capacitance, or any combination thereof.

The various illustrative logical blocks, modules, circuits, and algorithm steps described in connection with the arrangements disclosed herein may be implemented as electronic hardware, computer software, or combinations of both. To clearly illustrate this interchangeability of hardware and software, various illustrative components, blocks, modules, circuits, and steps have been described in terms of their functionality. Whether such functionality is implemented as hardware or software depends upon the particular application and design constraints imposed on the overall system. The described functionality may be implemented in varying ways for each particular application, but such implementation decisions should not be interpreted as causing a departure from the scope of the appended claims.

The various illustrative logical blocks, modules, circuits, and algorithm steps described in connection with the arrangements disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The actions of a method or algorithm described in connection with the arrangements disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other known form of storage medium. A storage medium may be coupled to the processor such that the processor can read information from, and write information to, the storage medium. In the alternative, the storage medium may be integral to the processor. The processor and the storage medium may reside in an ASIC. The ASIC may reside in functional equipment such as, e.g., a computer, a robot, a user terminal, a mobile telephone or tablet, a car, or an IP camera. In the alternative, the processor and the storage medium may reside as discrete components in such functional equipment. Additionally or in the alternative, at least one of the processor and/or the storage medium may reside in a cloud-based network such as, e.g., the Internet.

The above description is not intended to be exhaustive or to limit the features to the precise forms disclosed. Various alternatives and modifications can be devised without departing from the disclosure, and the generic principles defined herein may be applied to other arrangements without departing from the spirit or scope of the appended claims. Accordingly, the present disclosure is intended to embrace all such alternatives, modifications, and variations. Additionally, while several examples of the present disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as examples of particular configurations. And it should be envisioned that other modifications may be made within the scope and spirit of the claims appended hereto. Other elements, steps, actions, methods, and techniques that are not substantially different from those described above and/or in the appended claims are also intended to be within the scope of the disclosure. Thus, the appended claims are not intended to be limited to the examples shown and described herein, but are to be accorded the broadest scope consistent with the principles and novel features disclosed herein.

The examples shown in drawings are presented only to demonstrate certain examples of the disclosure. And, the drawings are merely illustrative and are non-limiting. In the drawings, for illustrative purposes, the size of some of the elements may be exaggerated and not drawn to a particular scale. Additionally, elements shown within the drawings that have the same numbers may be identical elements or may be similar elements, depending on the context.

Where the term “comprising” is used in the present description and claims, it does not exclude other elements or steps. Where an indefinite or definite article is used when referring to a singular noun, e.g., “a”, “an”, or “the”, this includes a plural of that noun unless something otherwise is specifically stated. Hence, the term “comprising” should not be interpreted as being restricted to the items listed thereafter; it does not exclude other elements or steps, and so the scope of the expression “a device comprising items A and B” should not be limited to devices consisting only of components A and B. Furthermore, to the extent that the terms “includes”, “has”, “possesses”, and the like are used in the present description and claims, such terms are intended to be inclusive in a manner similar to the term “comprising,” as “comprising” is interpreted when employed as a transitional word in a claim.

Furthermore, the terms “first”, “second”, “third”, and the like, whether used in the description or in the claims, are provided to distinguish between similar elements and not necessarily to describe a sequential or chronological order. It is to be understood that the terms so used are interchangeable under appropriate circumstances (unless clearly disclosed otherwise) and that the aspects of the disclosure described herein are capable of operation in other sequences and/or arrangements than are described or illustrated herein.

A system, comprising: a container arranged to store a plurality of inventory items at a plurality of storage sites within the container, each of the plurality of storage sites including a scale configured to generate a value defining the weight of the inventory item located at the storage site having the scale, the container further including at least one lockable door; a controller coupled to the container and configured to unlock the at least one lockable door, the controller being further coupled to the plurality of scales and configured to determine an identity, a weight, or a partial weight of any of the plurality of inventory items removed from or placed at the respective plurality of storage sites; and a security processor coupled to the controller and configured to attempt to authenticate a user of the system, and to inform the controller if the attempt to authenticate the user is successful, wherein the controller is configured to unlock the at least one door upon receiving information from the security processor that the attempt to authenticate was successful, and wherein the controller is further configured to alert the user of an error in removal or placement of any of the plurality of inventory items from or at any of the plurality of storage sites based on determined identities or weight values or partial weight values of inventory items.

The system of the preceding clause, further comprising at least one camera within the container configured to detect visual information about the plurality of inventory items at the plurality of storage sites, the camera being coupled to the controller, the controller being further configured to alert the user of an error in removal or placement of any of the plurality of inventory items from or at any of the plurality of storage sites based on the detected visual information.

The system of any preceding clause, further comprising an intelligence processor coupled to the controller and configured to compare the determined weight values or partial weight values received from the controller to prescription information for patients, and to place at least one order for at least one inventory item based on a result of the comparison.

The system of any preceding clause, further comprising an intelligence processor coupled to the controller and configured to assist the controller to determine the identities of any of the plurality of inventory items removed from or placed at any of the plurality of storage sites based on the determined weight values received from the controller.

The system of any preceding clause, further comprising an intelligence processor coupled to the controller and configured to determine an amount of a new order for at least one inventory item, and to place an order for at least one new inventory item.

The system of any preceding clause, wherein the intelligence processor is configured to determine the amount of the new order based on at least one of (i) existence of supply shortages for the at least one inventory item, (ii) an expiration date for the at least one inventory item, (iii) existence of a disease epidemic that the at least one inventory item can mitigate, (iv) pricing information associated with the at least one inventory item, (v) quantities of previous orders for the at least one inventory item, and (vi) seasonal information associated with the at least one inventory item.

The system of any preceding clause, wherein the intelligence processor is further configured to compare the determined weight values and partial weight values received from the controller to the prescription information to determine the identity of at least one of the plurality of inventory items.

The system of any preceding clause, wherein the security processor is configured to authenticate a user of the system based on at least one of (i) a wireless transmission, (ii) a keypad entry, (iii) a facial recognition, (iv) an RFID card scan, (v) a fingerprint recognition, and (vi) a gesture recognition.

A method, comprising: attempting, by a security processor, to authenticate a user of a system; unlocking, by a controller, a door of a container if the attempting step is successful; monitoring and storing, by the controller, removal or placement of inventory items at respective locations in the container by the user and weights or partial weights of the inventory items removed or placed; and alerting, by the controller, the user if any inventory item is removed from or placed at in incorrect location.

The method of the preceding clause, further comprising storing, by the controller, data captured by a camera in the container relating to placement or removal of inventory items from locations in the container.

The method of any preceding clause, further comprising comparing, by the controller and an intelligence processor, weights of removed inventory items or weights of partially removed inventory items to prescription data, and placing an order, by the intelligence processor, for at least one inventory item based on the comparing.

The method of any preceding clause, further comprising determining, by the intelligence processor, an amount of inventory items to order.

The method of any preceding clause, further comprising determining, by an intelligence processor, an identity of an inventory item removed from or placed at a location in the container based on the weight or partial weight of the inventory item.

The method of any preceding clause, wherein the attempting comprises attempting to authenticate the user of the system based on at least one of (i) a wireless transmission, (ii) a keypad entry, (iii) a facial recognition, (iv) an RFID card scan, (v) a fingerprint recognition, and (vi) a gesture recognition.

The method of any preceding clause, further comprising determining an amount of an inventory item to order and placing an order for the determined amount of the inventory item based on at least one of (i) existence of supply shortages for the inventory item, (ii) an expiration date for the inventory item, (iii) existence of a disease epidemic that the inventory item can mitigate, (iv) pricing information associated with the inventory item, (v) quantities of previous orders for the inventory item, and (vi) seasonal information associated with the inventory item.

A system, comprising: a scale; and a processor coupled to the scale and configured to obtain (i) a weight of an empty bin for storing a plurality of containers for storing items, (ii) a weight of an empty container, (iii) a weight of an item, and (iv) a maximum number of items storable in a container, the processor being further configured to obtain a first weight measurement for a bin sitting on the scale, the bin storing the plurality of containers, with one container of the plurality of containers storing a first number of items that is less than the maximum number of items storable in a container and the other containers of the plurality of containers each storing the maximum number of items storable in a container, the processor being further configured to subtract from the first weight measurement a sum of (i) the weight of an empty bin for storing a predefined number of containers for storing items, (ii) a product of a second number equal to one less than the plurality of containers for storing items and the weight of a container storing the maximum number of items storable in a container, and (iii) the weight of an empty container to generate therefrom a resultant weight value, and to divide the resultant weight value by the weight of an item to generate therefrom the first number of items.

The system of the preceding clause, wherein the processor is further configured to multiply the second number by the maximum number of items storable in a container to generate a count number of items, and to add the count number to the first number of items to generate a total number of items in the bin.

The system of any of preceding clause, wherein the processor is further configured to alert the system if the first number of items is not within a predefined fraction of an integer.

The system of any preceding clause, wherein the processor is further configured to obtain at least one previous total number of items and compare the total number of items in the bin to the at least one previous total number of items to generate a change in item count, compare the change in item count to at least one previous change in item count, and alert the system if the change in item count is greater than the at least one previous change in item count by more than a predefined threshold value.

The system of any preceding clause, wherein the processor is further configured to identify a user of the system who placed the bin on the scale to generate a first identity, compare the first identity to a second identity of a user who previously placed an order for the items, and alert the system if the first identity and the second identity are different.

The system of any preceding clause, wherein the processor is further configured to compare a count of items on the scale entered by a user of the system with the total number of items in the bin, and alert the system if the count of items on the scale entered by the user of the system and the total number of items in the bin are different.

The system of any preceding clause, wherein the processor is further configured to optimize quantity and supplier for an order of items by obtaining and evaluating at least one of a quantity of items in the system, at least one previous order of items, seasonal usage of items, available prices for items, available discounts for items, and available quantities of the items from suppliers, and to enable the order to be submitted by at least one of an automated system, a supplier API, and a manual entry by a user of the system.

The system of any preceding clause, wherein the processor is further configured to determine whether information about the items on the scale entered by a user of the system is recorded in the system, and to prompt the user of the system to enter the weight of a container, the weight of an item, and the maximum number of items storable in a container if the information about the items on the scale entered by a user of the system is not recorded in the system.

A system, comprising: a scale; and a processor coupled to the scale and configured to obtain at a first sampling rate a plurality of successive digital representations of a weight of an item sitting on the scale, filter the plurality of successive digital representations, down sample the filtered plurality of successive digital representations to a second sampling rate, the second sampling rate being lower than the first sampling rate, average the down sampled plurality of successive digital representations to generate a single digital representation, and apply a conversion factor of digital representations to weights in units to generate a weight in units of the item sitting on the scale.

The system of the preceding clause, wherein the conversion factor has been generated from previous weight measurements and stored by the system.

The system of any preceding clause, wherein the first sampling rate is predefined.

The system of any preceding clause, wherein the processor is configured to filter the plurality of successive digital representations to remove successive digital representations that differ by more than a threshold value from a metric associated with the plurality of successive digital representations.

The system of the preceding clause, wherein the metric is an average, and the threshold value is a predefined number of standard deviations.

The system of any preceding clause, wherein the processor is further configured to maintain a history of weight measurements of items, calculate a plurality of measurement periods, each measurement period having a start time and an end time, and in each measurement period a change in weight measurements being less than a predefined ceiling value, compare a weight measurement at an end of a measurement period with a weight measurement at a start of a next successive weight period to generate a relative weight change value.

The system of the preceding clause, wherein the processor is further configured to filter the history of weight measurements.

What is claimed is:

1. A system, comprising:
a container arranged to store a plurality of inventory items at a plurality of storage sites within the container, each of the plurality of storage sites including a scale configured to generate a value defining the weight of the inventory item located at the storage site having the scale, the container further including at least one lockable door;
a controller coupled to the container and configured to unlock the at least one lockable door, the controller being further coupled to the plurality of scales and configured to determine an identity, a weight, or a partial weight of any of the plurality of inventory items removed from or placed at the respective plurality of storage sites; and
a security processor coupled to the controller and configured to attempt to authenticate a user of the system, and to inform the controller if the attempt to authenticate the user is successful, wherein the controller is configured to unlock the at least one door upon receiving information from the security processor that the attempt to authenticate was successful, and wherein the controller is further configured to alert the user of an error in removal or placement of any of the plurality of inventory items from or at any of the plurality of storage sites based on determined identities or weight values or partial weight values of inventory items.

2. The system of claim 1, further comprising at least one camera within the container configured to detect visual information about the plurality of inventory items at the plurality of storage sites, the camera being coupled to the controller, the controller being further configured to alert the user of an error in removal or placement of any of the plurality of inventory items from or at any of the plurality of storage sites based on the detected visual information.

3. The system of claim 1, further comprising an intelligence processor coupled to the controller and configured to compare the determined weight values or partial weight values received from the controller to prescription information for patients, and to place at least one order for at least one inventory item based on a result of the comparison.

4. The system of claim 1, further comprising an intelligence processor coupled to the controller and configured to assist the controller to determine the identities of any of the plurality of inventory items removed from or placed at any of the plurality of storage sites based on the determined weight values received from the controller.

5. The system of claim 1, further comprising an intelligence processor coupled to the controller and configured to determine an amount of a new order for at least one inventory item, and to place an order for at least one new inventory item.

6. The system of claim 5, wherein the intelligence processor is configured to determine the amount of the new order based on at least one of (i) existence of supply shortages for the at least one inventory item, (ii) an expiration date for the at least one inventory item, (iii) existence of a disease epidemic that the at least one inventory item can mitigate, (iv) pricing information associated with the at least one inventory item, (v) quantities of previous orders for the at least one inventory item, and (vi) seasonal information associated with the at least one inventory item.

7. The system of claim 3, wherein the intelligence processor is further configured to compare the determined weight values and partial weight values received from the controller to the prescription information to determine the identity of at least one of the plurality of inventory items.

8. The system of claim 1; wherein the security processor is configured to authenticate a user of the system based on at least one of (i) a wireless transmission, (ii) a keypad entry, (iii) a facial recognition, (iv) an RFID card scan, (v) a fingerprint recognition, and (vi) a gesture recognition.

9. A method, comprising:
attempting, by a security processor, to authenticate a user of a system;
unlocking, by a controller, a door of a container if the attempting step is successful;
monitoring and storing, by the controller, removal or placement of inventory items at respective locations in the container by the user and weights or partial weights of the inventory items removed or placed; and
alerting, by the controller, the user if any inventory item is removed from or placed at in incorrect location.

10. The method of claim 9, further comprising storing, by the controller, data captured by a camera in the container relating to placement or removal of inventory items from locations in the container.

11. The method of claim 9, further comprising comparing, by the controller and an intelligence processor, weights of removed inventory items or weights of partially removed inventory items to prescription data, and placing an order, by the intelligence processor, for at least one inventory item based on the comparing.

12. The method of claim 11, further comprising determining, by the intelligence processor, an amount of inventory items to order.

13. The method of claim 9, further comprising determining, by an intelligence processor, an identity of an inventory item removed from or placed at a location in the container based on the weight or partial weight of the inventory item.

14. The method of claim 9, wherein the attempting comprises attempting to authenticate the user of the system based on at least one of (i) a wireless transmission, (ii) a keypad entry, (iii) a facial recognition, (iv) an RFID card scan, (v) a fingerprint recognition, and (vi) a gesture recognition.

15. The method of claim 9, further comprising determining an amount of an inventory item to order and placing an order for the determined amount of the inventory item based on at least one of (i) existence of supply shortages for the inventory item, (ii) an expiration date for the inventory item, (iii) existence of a disease epidemic that the inventory item can mitigate, (iv) pricing information associated with the inventory item, (v) quantities of previous orders for the inventory item, and (vi) seasonal information associated with the inventory item.

16. A system, comprising:
a scale; and
a processor coupled to the scale and configured to obtain (i) a weight of an empty bin for storing a plurality of containers for storing items, (ii) a weight of an empty container, (iii) a weight of an item, and (iv) a maximum number of items storable in a container,
the processor being further configured to obtain a first weight measurement for a bin sitting on the scale, the bin storing the plurality of containers, with one container of the plurality of containers storing a first number of items that is less than the maximum number of items storable in a container and the other containers of the plurality of containers each storing the maximum number of items storable in a container,
the processor being further configured to subtract from the first weight measurement a sum of (i) the weight of an empty bin for storing a predefined number of containers for storing items, (ii) a product of a second number equal to one less than the plurality of containers for storing items and the weight of a container storing the maximum number of items storable in a container, and (iii) the weight of an empty container to generate therefrom a resultant weight value, and to divide the resultant weight value by the weight of an item to generate therefrom the first number of items.

17. The system of claim 16, wherein the processor is further configured to multiply the second number by the maximum number of items storable in a container to generate a count number of items, and to add the count number to the first number of items to generate a total number of items in the bin.

18. The system of claim 16, wherein the processor is further configured to alert the system if the first number of items is not within a predefined fraction of an integer.

19. The system of claim 16, wherein the processor is further configured to obtain at least one previous total number of items and compare the total number of items in the bin to the at least one previous total number of items to generate a change in item count, compare the change in item count to at least one previous change in item count, and alert the system if the change in item count is greater than the at least one previous change in item count by more than a predefined threshold value.

20. The system of claim 16, wherein the processor is further configured to identify a user of the system who placed the bin on the scale to generate a first identity, compare the first identity to a second identity of a user who previously placed an order for the items, and alert the system if the first identity and the second identity are different.

21. The system of claim 16, wherein the processor is further configured to compare a count of items on the scale entered by a user of the system with the total number of items in the bin, and alert the system if the count of items on the scale entered by the user of the system and the total number of items in the bin are different.

22. The system of claim 16, wherein the processor is further configured to optimize quantity and supplier for an order of items by obtaining and evaluating at least one of a quantity of items in the system, at least one previous order of items, seasonal usage of items, available prices for items, available discounts for items, and available quantities of the items from suppliers, and to enable the order to be submitted by at least one of an automated system, a supplier API, and a manual entry by a user of the system.

23. The system of claim 16, wherein the processor is further configured to determine whether information about the items on the scale entered by a user of the system is recorded in the system, and to prompt the user of the system to enter the weight of a container, the weight of an item, and the maximum number of items storable in a container if the information about the items on the scale entered by a user of the system is not recorded in the system.

24. A system, comprising:
a scale; and
a processor coupled to the scale and configured to obtain at a first sampling rate a plurality of successive digital representations of a weight of an item sitting on the scale, filter the plurality of successive digital representations, down sample the filtered plurality of successive digital representations to a second sampling rate, the second sampling rate being lower than the first sampling rate, average the down sampled plurality of successive digital representations to generate a single digital representation, and apply a conversion factor of digital representations to weights in units to generate a weight in units of the item sitting on the scale.

25. The system of claim 24, wherein the conversion factor has been generated from previous weight measurements and stored by the system.

26. The system of claim 24, wherein the first sampling rate is predefined.

27. The system of claim 24, wherein the processor is configured to filter the plurality of successive digital representations to remove successive digital representations that differ by more than a threshold value from a metric associated with the plurality of successive digital representations.

28. The system of claim 27, wherein the metric is an average, and the threshold value is a predefined number of standard deviations.

29. The system of claim 24, wherein the processor is further configured to maintain a history of weight measurements of items, calculate a plurality of measurement periods, each measurement period having a start time and an end time, and in each measurement period a change in weight measurements being less than a predefined ceiling value, compare a weight measurement at an end of a measurement period with a weight measurement at a start of a next successive weight period to generate a relative weight change value.

30. The system of claim 29, wherein the processor is further configured to filter the history of weight measurements.
